(19) United States
(12) Patent Application Publication
(10) Pub. No.: US 2022/0361769 A1
(43) Pub. Date: Nov. 17, 2022

(54) LOAD MEASUREMENT SYSTEM, WALKING TRAINING SYSTEM, LOAD MEASUREMENT METHOD, AND PROGRAM

(71) Applicant: TOYOTA JIDOSHA KABUSHIKI KAISHA, Toyota-shi (JP)

(72) Inventor: Yo SATO, Miyoshi-shi (JP)

(21) Appl. No.: 17/655,760

(22) Filed: Mar. 21, 2022

(30) Foreign Application Priority Data
May 12, 2021 (JP) 2021-081174

(51) Int. Cl. A61B 5/103; A61B 5/11

(52) U.S. Cl. CPC A61B 5/1036; A61B 5/112; A61B 5/1122

(57) ABSTRACT

A load measurement device includes an acquisition unit, a first extraction unit, a second extraction unit, and an output unit. The acquisition unit acquires measurement information output from a load distribution sensor that detects a load distribution received from a sole of a subject. The first extraction unit extracts a region having a load value equal to or larger than a first threshold value as a first region based on the measurement information, and detects the first region as a sole region. The second extraction unit extracts a second region having a load value equal to or larger than a second threshold value that is smaller than the first threshold value from a re-extraction target region defined with the first region as a reference, and adds the second region to the sole region. The output unit outputs information related to a load distribution of the sole region.

### CROSS-REFERENCE TO RELATED APPLICATION

This application claims priority to Japanese Patent Application No. 2021-081174 filed on May 12, 2021, incorporated herein by reference in its entirety.

### BACKGROUND

### 1. Technical Field

The present disclosure relates to a load measurement system, a walking training system, a load measurement method, and a program.

### 2. Description of Related Art

A walking training system has been developed for rehabilitation (hereinafter referred to as “rehab”) patients to train their walking motion. In the walking training system, a load distribution of the trainee is measured by a load distribution sensor installed on the treadmill. For example, WO 2006/106714 discloses a pressure distribution detection device including two types of loop electrode groups, an elastic body on the loop electrode groups, and a conductive substance on the elastic body. The walking training system calculates a sole region from the load distribution of the measurement result, estimates the walking state of the trainee based on the load distribution-related information that is information related to the load distribution of the sole region, and assists the motion of joints of the trainee.

### SUMMARY

The walking training system detects a region, out of the load distribution, having a load equal to or higher than a detection threshold value, as the sole region of the trainee. Here, when the detection threshold value is set high, there is a problem that a region that is not detected as the sole region is generated and the load distribution-related information of the sole region cannot be obtained correctly. For example, when the walking training system calculates a center of pressure (COP) of the sole region as the load distribution information, the COP may shift backward because the low load region corresponding to the toe or the like is not detected. When the body weight of the trainee is light or when the load received from the sole of one leg is less than the load received from the sole of the other leg due to paralysis, the problem mentioned above is serious because the ratio of the low load region with respect to the load distribution is high.

In contrast, when the detection threshold value is set low, there is a problem that the load distribution-related information of the sole region cannot be obtained correctly if noise is detected, because of the influence of the noise.

The present disclosure has been made to solve such problems, and provides a load measurement system, a walking training system, a load measurement method, and a program that improve the measurement accuracy of the load distribution-related information of the sole region.

A load measurement system according to a first aspect of the present disclosure includes an acquisition unit, a first extraction unit, a second extraction unit, and an output unit. The acquisition unit acquires measurement information output from a load distribution sensor that detects a load distribution received from a sole of a subject. The first extraction unit extracts a region having a load value equal to or larger than a first threshold value as a first region based on the measurement information, and detects the first region as a sole region. The second extraction unit extracts a second region having a load value equal to or larger than a second threshold value that is smaller than the first threshold value from a re-extraction target region defined with the first region as a reference, and adds the second region to the sole region. The output unit outputs information related to a load distribution of the sole region. This enables the load measurement system to accurately detect a low load region such as a toe while reducing the influence of noise. Thus, the measurement accuracy of the load distribution-related information of the sole region is improved.

The load measurement system may further include a calculation unit that calculates a center of pressure of the sole region. The output unit may output information on the center of pressure. This enables the load measurement system to reduce errors in the position of the center of pressure.

The second extraction unit may determine, as the re-extraction target region, a region located within a predetermined distance from a position of the first region. By limiting the re-extraction target region to the region close to the first region (high load region), the influence of noise in the distant region can be reduced. Further, the processing load of re-extraction is reduced, so it is possible to detect the sole region in real time.

The second extraction unit may determine the re-extraction target region such that a front end portion of the re-extraction target region in a walking direction protrudes forward in the walking direction of the subject by a predetermined distance from a front end portion of the first region in the walking direction. This enables the load measurement system to easily detect a low load region corresponding to the toe.

The second extraction unit may determine the re-extraction target region such that a left end portion of the re-extraction target region protrudes to left by a predetermined distance from a left end portion of the first region when the first region is a sole region of a right leg. The second extraction unit may determine the re-extraction target region such that a right end portion of the re-extraction target region protrudes to right by a predetermined distance from a right end portion of the first region when the first region is a sole region of a left leg. This enables the load measurement system to easily detect a low load region corresponding to the arch of the foot.

The second extraction unit may determine the re-extraction target region based on position information of a rear end portion of the sole region in a walking direction at a standing initial phase and foot length information of the subject. This makes it possible to determine the re-extraction target region from the foot length information with the portion that the heel of the subject first comes into contact, so that the estimation accuracy of the sole region is improved.

The second extraction unit may set the second threshold value based on attribute information of the subject. This enables the load measurement system to acquire a suitable load measurement result for each subject, and therefore the measurement accuracy is improved.

The second extraction unit may change the second threshold value based on a history of an area or a shape of the sole region during walking of the subject. This makes it possible to suitably perform the measurement even for the subject whose gait is not stable, and the measurement accuracy is improved.

A walking training system according to a second aspect of the present disclosure includes: the load distribution sensor; the load measurement system; and a control device that controls extension of a leg robot attached to at least one leg of the subject based on the information related to the load distribution of the sole region of the subject. This enables the walking training system to suitably control the leg robot and carry out suitable walking training.

A load measurement method according to a third aspect of the present disclosure includes: a step of acquiring measurement information output from a load distribution sensor that detects a load distribution received from a sole of a subject; a step of extracting a region having a load value equal to or larger than a first threshold value as a first region based on the measurement information, and detecting the first region as a sole region; a step of extracting a second region having a load value equal to or larger than a second threshold value that is smaller than the first threshold value from a re-extraction target region defined with the first region as a reference, and adding the second region to the sole region; and a step of outputting information related to a load distribution of the sole region. This makes it possible to accurately detect a low load region such as a toe while reducing the influence of noise. Thus, the measurement accuracy of the load distribution-related information of the sole region is improved.

A program according to a fourth aspect of the present disclosure causes a computer to execute a load measurement method. The load measurement method includes: a step of acquiring measurement information output from a load distribution sensor that detects a load distribution received from a sole of a subject; a step of extracting a region having a load value equal to or larger than a first threshold value as a first region based on the measurement information, and detecting the first region as a sole region; a step of extracting a second region having a load value equal to or larger than a second threshold value that is smaller than the first threshold value from a re-extraction target region defined with the first region as a reference, and adding the second region to the sole region; and a step of outputting information related to a load distribution of the sole region. This makes it possible to accurately detect a low load region such as a toe while reducing the influence of noise. Thus, the measurement accuracy of the load distribution-related information of the sole region is improved.

The present disclosure can provide a load measurement system, a walking training system, a load measurement method, and a program with which the measurement accuracy of the load distribution-related information of the sole region is improved.

### BRIEF DESCRIPTION OF THE DRAWINGS

Features, advantages, and technical and industrial significance of exemplary embodiments of the disclosure will be described below with reference to the accompanying drawings, in which like signs denote like elements, and wherein:

FIG. 1 is a schematic perspective view of a walking training system according to a first embodiment;

FIG. 2 is a schematic perspective view showing a configuration example of a walking assist device;

FIG. 3 shows a side view and a top view of a treadmill according to the first embodiment;

FIG. 4 is a block diagram showing a schematic configuration of a load measurement device according to the first embodiment;

FIG. 5 is a diagram illustrating a first region according to the first embodiment;

FIG. 6 is a diagram illustrating an example of a re-extraction target region according to the first embodiment;

FIG. 7 is a diagram illustrating an example of the re-extraction target region according to the first embodiment;

FIG. 8 is a diagram illustrating an example of the re-extraction target region according to the first embodiment;

FIG. 9 is a flowchart showing a procedure of a load measurement method according to the first embodiment;

FIG. 10 is a diagram illustrating an example of a re-extraction target region according to a second embodiment;

FIG. 11 is a flowchart showing a procedure of a load measurement method according to a third embodiment; and

FIG. 12 is a schematic configuration diagram of a computer that is used as the load measurement device and a system control unit according to the first to third embodiments.

### DETAILED DESCRIPTION OF EMBODIMENTS

Hereinafter, the present disclosure will be described through embodiments, but the disclosure according to the claims is not limited to the following embodiments. Moreover, not all of the configurations described in the embodiments are indispensable as means for solving the problem. For the sake of clarity, omission and simplification are made in the following description and drawings as appropriate.

### First Embodiment

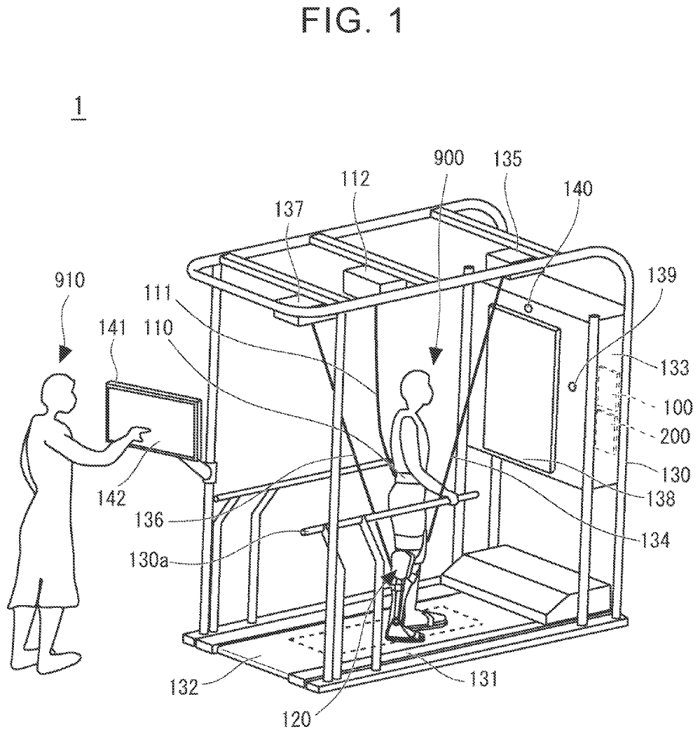

Hereinafter, a first embodiment of the present disclosure will be described. FIG. 1 is a schematic perspective view of a walking training system 1 according to the first embodiment. The walking training system 1 is an example of a system to which a load measurement device (also referred to as a load measurement system) according to the first embodiment can be applied. The walking training system 1 is a system for a trainee 900 who is a hemiplegic patient suffering from paralysis in one leg to perform walking training. The trainee 900 is also referred to as a subject. The up-down direction, the right-left direction, and the front-rear direction in the following description represent directions with the direction of the trainee 900 as a reference.

The walking training system 1 mainly includes a control panel 133 attached to a frame 130 constituting the entire skeleton, a treadmill 131 on which the trainee 900 walks, and a walking assist device 120 attached to at least one leg of the trainee 900. In the first embodiment, at least one leg is the affected leg that is the leg of the trainee 900 on the paralyzed side.

The frame 130 is provided to stand on a treadmill 131 installed on the floor. The treadmill 131 rotates a ring-shaped belt 132 with a motor (not shown). Thus, the belt 132 travels along the orbit. The treadmill 131 is a device that prompts the trainee 900 to walk. The trainee 900 who performs walking training rides on the belt 132 and attempts a walking motion with respect to the walking surface provided on the belt 132.

The frame 130 supports the control panel 133, a training monitor 138, and an audio output unit 139. The control panel 133 accommodates a load measurement device 100 and a system control unit 200. The load measurement device 100 is a computer device that measures information related to the load distribution of the sole region of the trainee 900 based on the measurement result of the sensor. The information related to the load distribution is also referred to as load distribution-related information. The system control unit 200 is also referred to as a control device, and is a computer device that controls sensors and motors. For example, the system control unit 200 controls the extension of the walking assist device 120 based on the load distribution-related information of the sole region of the trainee 900 that is measured by the load measurement device 100.

The training monitor 138 is a display device that presents information on training and measurement to the trainee 900. The training monitor 138 is, for example, a liquid crystal panel. The training monitor 138 is installed such that the trainee 900 can visually recognize the training monitor 138 while walking on the belt 132 of the treadmill 131.

The audio output unit 139 outputs information on training and measurement by voice and notifies the trainee 900. The audio output unit 139 is, for example, a speaker. The audio output unit 139 is installed at a position where the trainee 900 can hear while walking on the belt 132 of the treadmill 131.

Further, the frame 130 supports a front tension unit 135 at the front of the overhead portion of the trainee 900, a harness tension unit 112 at the overhead portion, and a rear tension unit 137 at the rear of the overhead portion. The frame 130 may include handrails 130a for the trainee 900 to grab.

The camera 140 is a front camera unit that takes an image of the trainee 900 at such an angle of view that the gait of the trainee 900 can be recognized from the front. The camera 140 may include a side camera unit that takes an image of the trainee 900 at such an angle of view that the gait of the trainee 900 can be recognized from the side. The camera 140 in the present embodiment includes a set of a lens and an imaging element that provides such an angle of view that the whole body including the head of the trainee 900 standing on the belt 132 can be captured. The imaging element is, for example, a complementary metal oxide semiconductor (CMOS) image sensor, and converts an optical image on an image plane into an image signal. The camera 140 is installed near the training monitor 138 so as to face the trainee 900. When the camera 140 includes the side camera unit, the side camera unit may be installed on the handrail 130a so as to capture the trainee 900 from the side.

One end of a front wire 134 is connected to a winding mechanism of the front tension unit 135, and the other end is connected to the walking assist device 120. The winding mechanism of the front tension unit 135 winds and unwinds the front wire 134 in accordance with the movement of the affected leg, by turning on and off a motor (not shown) following the instruction of the system control unit 200. Similarly, one end of a rear wire 136 is connected to a winding mechanism of the rear tension unit 137, and the other end is connected to the walking assist device 120. The winding mechanism of the rear tension unit 137 winds and unwinds the rear wire 136 in accordance with the movement of the affected leg by turning on and off a motor (not shown) following the instruction of the system control unit 200. With such a coordinated operation of the front tension unit 135 and the rear tension unit 137, the load of the walking assist device 120 is offset so as not to be a burden on the affected leg, and further, the forward swing motion of the affected leg is assisted in accordance with the degree of the setting.

An operator 910 who is a training assistant sets the assist level high, for a trainee who has severe paralysis. The operator 910 is a physiotherapist or a doctor who has the authority to select, correct, and add the setting items of the walking training system 1. When the assist level is set high, the front tension unit 135 winds up the front wire 134 with a relatively large force in accordance with the forward swing timing of the affected leg. As the training progresses and assistance becomes no longer needed, the operator sets the assist level to the minimum. When the assist level is set to the minimum, the front tension unit 135 winds up the front wire 134 with a force to cancel the weight of the walking assist device 120 in accordance with the forward swing timing of the affected leg.

The walking training system 1 includes a safety device including, as main components, a safety brace 110, a harness wire 111, and the harness tension unit 112. The safety brace 110 is a belt wrapped around the abdomen of the trainee 900 and is fixed to the waist portion by, for example, a hook-and-loop fastener. One end of the harness wire 111 is connected to the safety brace 110, and the other end is connected to the winding mechanism of the harness tension unit 112. The winding mechanism of the harness tension unit 112 winds and unwinds the harness wire 111 by turning on and off a motor (not shown). With such a configuration, when the trainee 900 significantly loses his/her posture, the safety device winds up the harness wire 111 following the instruction of the system control unit 200 that has detected the movement, and supports the upper body of the trainee 900 with the safety brace 110.

A management monitor 141 is attached to the frame 130 and is a display device for the operator 910 to monitor and operate. The management monitor 141 is, for example, a liquid crystal panel, and a touch panel is superimposed on the surface of the management monitor 141 as an example of an input portion 142. The management monitor 141 presents various menu items related to settings of training and measurement, various parameter values at the time of training and measurement, measurement results during training, and the like. The operator 910 selects, corrects, or adds a setting item via the input portion 142 such as a touch panel or a keyboard (not shown). The management monitor 141 is installed at a position where the trainee 900 cannot visually recognize the display from the training trial position on the treadmill 131. A support portion that supports the management monitor 141 may have a rotation mechanism that inverts the display surface in order to cope with the case where the operator 910 intentionally shows the display screen to the trainee 900.

The walking assist device 120 is attached to the affected leg of the trainee 900 and assists the trainee 900 in walking by reducing the load of extension and bending on the knee joint of the affected leg. The walking assist device 120 transmits data on the leg movement acquired through walking training to the system control unit 200, or drives the joint portion following the instruction from the system control unit 200. The walking assist device 120 can also be connected to a hip joint (a connecting member including a rotating portion) attached to the safety brace 110 that is a part of the fall prevention harness device via a wire or the like.

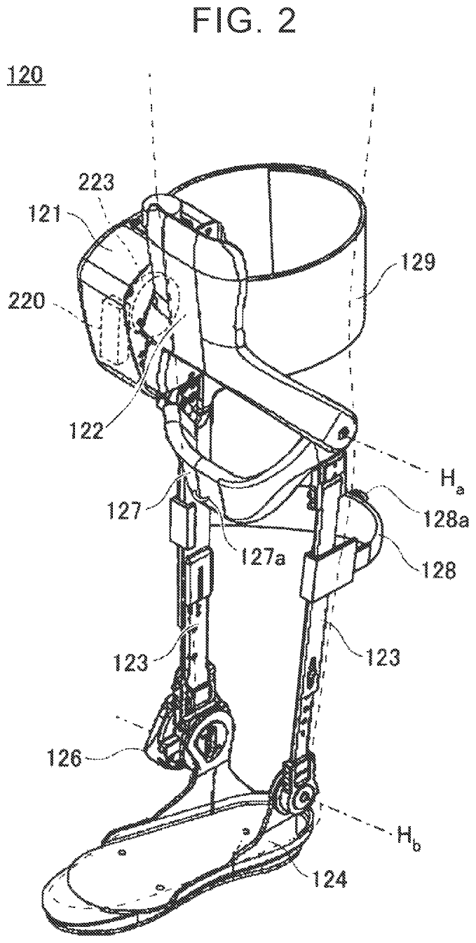

FIG. 2 is a schematic perspective view showing a configuration example of the walking assist device 120. The walking assist device 120 mainly includes a control unit 121 and a plurality of frames that supports various parts of the affected leg. The walking assist device 120 is also referred to as a leg robot.

The control unit 121 includes an auxiliary control unit 220 that controls the walking assist device 120, and also includes a motor (not shown) that generates a driving force for assisting the extension motion and the bending motion of the knee joint. The frames that support various parts of the affected leg include an upper leg frame 122 and lower leg frames 123 that are pivotably connected to the upper leg frame 122. The frames further include a foot flat frame 124 pivotably connected to the lower leg frames 123, a front connecting frame 127 for connecting the front wire 134, and a rear connecting frame 128 for connecting the rear wire 136.

The upper leg frame 122 and the lower leg frames 123 pivot relative to each other around a hinge axis Hₐ shown in the figure. The motor of the control unit 121 rotates following the instruction of the auxiliary control unit 220 to force the upper leg frame 122 and the lower leg frames 123 to relatively open or close around the hinge axis Hₐ. The angle sensor 223 housed in the control unit 121 is, for example, a rotary encoder, and detects the angle formed by the upper leg frame 122 and the lower leg frames 123 around the hinge axis Hₐ. The lower leg frames 123 and the foot flat frame 124 pivot relative to each other around the hinge axis H_{b} shown in the figure. The relative pivot angle range is adjusted in advance by an adjusting mechanism 126.

The front connecting frame 127 is provided so as to extend in the right-left direction on the front side of the upper leg and connect to the upper leg frame 122 at both ends. The front connecting frame 127 is further provided with a connecting hook 127a for connecting the front wire 134, near the center in the right-left direction. The rear connecting frame 128 is provided so as to extend in the right-left direction on the rear side of the lower leg and connect to the lower leg frames 123 extending in the up-down direction at both ends. The rear connecting frame 128 is further provided with a connecting hook 128a for connecting the rear wire 136, near the center in the right-left direction.

The upper leg frame 122 includes an upper leg belt 129. The upper leg belt 129 is a belt integrally provided on the upper leg frame, and is wrapped around the upper leg portion of the affected leg to fix the upper leg frame 122 to the upper leg portion. This suppresses the entire walking assist device 120 from shifting with respect to the legs of the trainee 900.

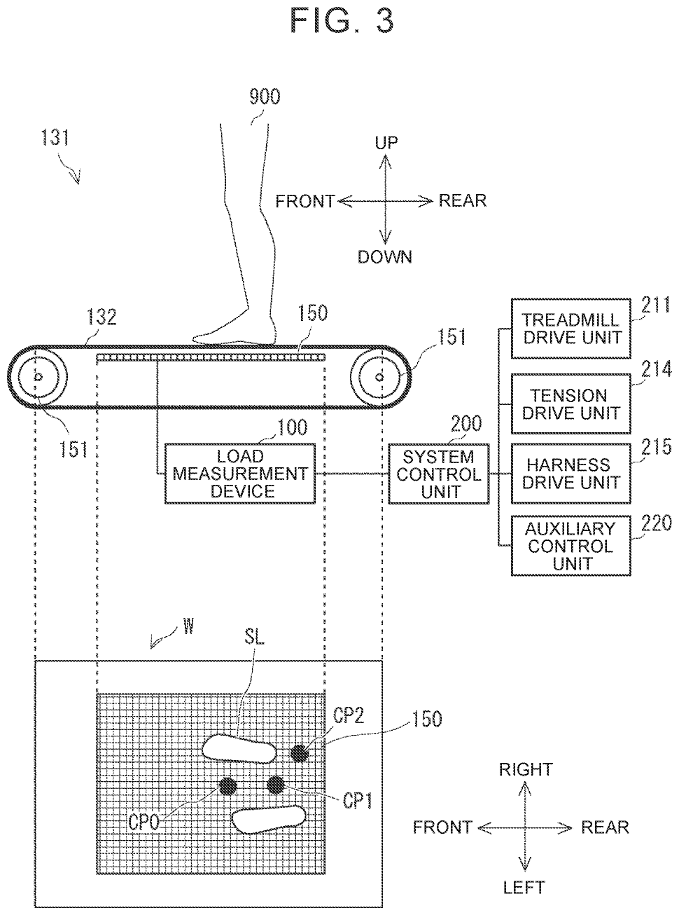

FIG. 3 shows a side view and a top view of the treadmill according to the first embodiment. The treadmill 131 includes at least a ring-shaped belt 132, a pulley 151, and a motor (not shown).

The load distribution sensor 150 is disposed inside the belt 132, that is, on the side of the belt 132 opposite to the surface on which the trainee 900 rides. The load distribution sensor 150 is fixed to the body of the treadmill 131 so as not to move together with the belt 132.

The load distribution sensor 150 is a load distribution sensor sheet with a plurality of pressure detection points. The pressure detection points are arranged in a matrix so as to be parallel with a walking surface W (mounting surface) that supports the sole of the trainee 900 in the standing state. Further, the load distribution sensor 150 is disposed toward the center of the walking surface W in the right-left direction that is orthogonal to the walking front-rear direction. The walking front-rear direction is a direction parallel to the traveling direction of the belt 132. By using output values of the pressure detection points, the load distribution sensor 150 can detect the magnitude and the distribution of the vertical load received from the sole of the trainee 900. Thereby, the load distribution sensor 150 detects the position of the sole of the trainee 900 in the standing state and the distribution of the load received from the sole of the trainee 900, via the belt 132. Here, the ground contact region of the sole is referred to as a sole region SL. The position of the sole region SL is also referred to as a standing position or a stepping position of the trainee 900.

The load distribution sensor 150 is connected to the load measurement device 100. The load measurement device 100 acquires measurement information output from the load distribution sensor 150, and calculates the load distribution-related information of the sole region of the trainee 900 based on the measurement information. The load measurement device 100 supplies the measured load distribution-related information of the sole region to the system control unit 200.

The system control unit 200 controls various drive units based on the load distribution-related information of the sole region. For example, the system control unit 200 is connected to a treadmill drive unit 211, a tension drive unit 214, a harness drive unit 215, and an auxiliary control unit 220 of the walking assist device 120 by wire or wirelessly. The system control unit 200 transmits drive signals to the treadmill drive unit 211, the tension drive unit 214, and the harness drive unit 215, and transmits a control signal to the auxiliary control unit 220.

The treadmill drive unit 211 includes the above motor for rotating the belt 132 of the treadmill 131 and a drive circuit thereof. The system control unit 200 performs rotation control of the belt 132 by transmitting a drive signal to the treadmill drive unit 211. The system control unit 200 adjusts the rotation speed of the belt 132 in accordance with, for example, the walking speed set by the operator 910. Alternatively, the system control unit 200 adjusts the rotation speed of the belt 132 in accordance with the load distribution-related information of the sole region output from the load measurement device 100.

The tension drive unit 214 includes a motor for tensioning the front wire 134 and a drive circuit thereof that are provided in the front tension unit 135, and a motor for tensioning the rear wire 136 and a drive circuit thereof that are provided in the rear tension unit 137. The system control unit 200 controls the winding of the front wire 134 and the winding of the rear wire 136 by transmitting a drive signal to the tension drive unit 214. Further, the system control unit 200 controls the tensile force of each wire by controlling the driving torque of the motor, not limited to the winding operation. Further, the system control unit 200 identifies the timing at which the affected leg switches from the standing state to the swinging state in accordance with the load distribution-related information of the sole region output from the load measurement device 100, and increases or decreases the tensile force of each wire in synchronization with that timing, thereby assisting the motion of the affected leg.

The harness drive unit 215 includes a motor for tensioning the harness wire 111 and a drive circuit thereof that are provided in the harness tension unit 112. The system control unit 200 controls the winding of the harness wire 111 and the tensile force of the harness wire 111 by transmitting a drive signal to the harness drive unit 215. For example, when the trainee 900 is predicted to fall, the system control unit 200 winds up the harness wire 111 by a certain amount to suppress the trainee from falling.

The auxiliary control unit 220 is, for example, a microprocessor unit (MPU), and performs control of the walking assist device 120 by executing a control program provided from the system control unit 200. Moreover, the auxiliary control unit 220 notifies the system control unit 200 of the state of the walking assist device 120. Furthermore, the auxiliary control unit 220 receives a command from the system control unit 200 based on the load distribution-related information of the sole region and performs control of starting, stopping, and the like of the walking assist device 120.

The auxiliary control unit 220 transmits a drive signal to the joint drive unit including the motor of the control unit 121 and the drive circuit thereof, to force the upper leg frame 122 and the lower leg frames 123 to relatively open or close around the hinge axis Hₐ. Such motions assist the knee extension and bending motions and suppress knee collapse. The auxiliary control unit 220 receives a detection signal from an angle sensor (not shown) that detects the angle formed by the upper leg frame 122 and the lower leg frames 123 around the hinge axis Hₐ, and calculates the opening angle of the knee joint.

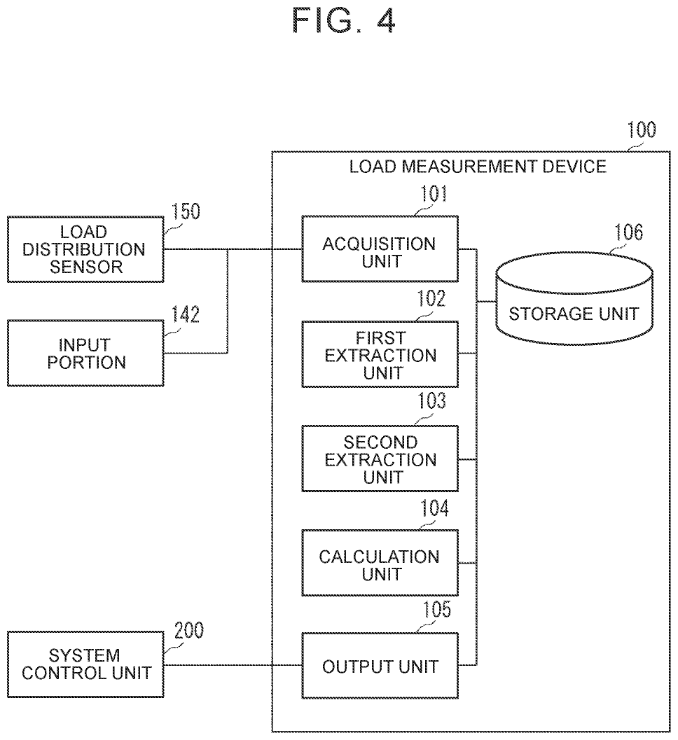

FIG. 4 is a block diagram showing a schematic configuration of the load measurement device 100 according to the first embodiment. The load measurement device 100 includes an acquisition unit 101, a first extraction unit 102, a second extraction unit 103, a calculation unit 104, an output unit 105, and a storage unit 106. The components of the load measurement device 100 are connected to each other.

The acquisition unit 101 acquires the measurement information output from the load distribution sensor 150. The measurement information includes load value information corresponding to each of the pressure detection points at different positions. For example, the acquisition unit 101 is connected to the load distribution sensor 150 and acquires measurement information from the load distribution sensor 150. Alternatively, the acquisition unit 101 may be connected to another device (not shown) connected to the load distribution sensor 150 and acquire measurement information from the other device. The acquisition unit 101 supplies the measurement information to the first extraction unit 102, the second extraction unit 103, and the calculation unit 104.

The acquisition unit 101 is also connected to the input portion 142 and acquires the input information received by the input portion 142. In the first embodiment, the input information may include first input information for determining a first threshold value, second input information for determining a second threshold value, and third input information for determining a re-extraction target region, which will be described below. In the first embodiment, the first and second input information is a weight value of the trainee 900. However, the first and second input information may include, in place of or in addition to the weight value, other attribute information of the trainee 900, such as gender, age, foot length information, and rehab stage level of the trainee 900. The third input information is information indicating the type of the re-extraction target. The re-extraction target may be a low load region such as “toe”, “arch of the foot” or “heel”. The third information may include, in place of or in addition to the type of the re-extraction target, attribute information of the trainee 900, such as gender, age, foot length information, and rehab stage level of the trainee 900. The acquisition unit 101 supplies the first input information to the first extraction unit 102, and supplies the second input information and the third input information to the second extraction unit 103.

The first extraction unit 102 sets the first threshold value based on the first input information. The first extraction unit 102 extracts, as the first region, a region having a load value equal to or larger than a predetermined first threshold value based on the measurement information. The first region is sometimes referred to as a high load region. The first extraction unit 102 provisionally detects the extracted first region as the sole region SL. The first extraction unit 102 supplies the position information of the first region detected as the sole region SL to the second extraction unit 103.

The second extraction unit 103 determines a region defined with the first region as a reference, as the re-extraction target region. Specifically, the second extraction unit 103 determines the re-extraction target region based on the position information of the first region and the third input information. Here, the second extraction unit 103 may determine a region located within a predetermined distance from the position of the first region as the re-extraction target region. That is, the re-extraction target region may be a region close to the high load region. By limiting the re-extraction target region to the region close to the high load region, the influence of noise in the distant region can be reduced. Further, the processing load of re-extraction is reduced, so it is possible to detect the sole region SL in real time.

Further, the second extraction unit 103 sets the second threshold value based on the first threshold value and the second input information. The second threshold value is a value smaller than the first threshold value. When the re-extraction target region includes a region having a load value equal to or larger than the predetermined second threshold value, the second extraction unit 103 extracts the region from the re-extraction target region, as the second region. The second region is sometimes referred to as a low load region. The second extraction unit 103 adds the second region to the sole region SL. That is, when the re-extraction target region includes the second region, the sole region SL is a region including the first region and the second region, for example, a region of the union portion of the first region and the second region. In contrast, when the re-extraction target region does not include the second region, the sole region SL is a region including the first region and may coincide with the first region. The second extraction unit 103 supplies the position information of the sole region SL to the calculation unit 104.

The calculation unit 104 calculates the load distribution-related information of the sole region SL based on the position information and the measurement information of the sole region SL. In the first embodiment, the load distribution-related information of the sole region SL is a center of pressure (COP) of the sole region SL. However, in place of or in addition to this, the calculation unit 104 may calculate the sum of the load values of the sole region SL or may generate the load distribution information of the sole region SL from the measurement information, as the load distribution-related information of the sole region SL. The calculation unit 104 supplies the load distribution-related information of the sole region SL to the output unit 105.

The output unit 105 outputs the load distribution-related information of the sole region SL to the system control unit 200. In the first embodiment, the output unit 105 outputs information on the COP, for example, the position information of the COP.

The storage unit 106 is a storage medium for storing information necessary for processing of the load measurement device 100 and the generated information.

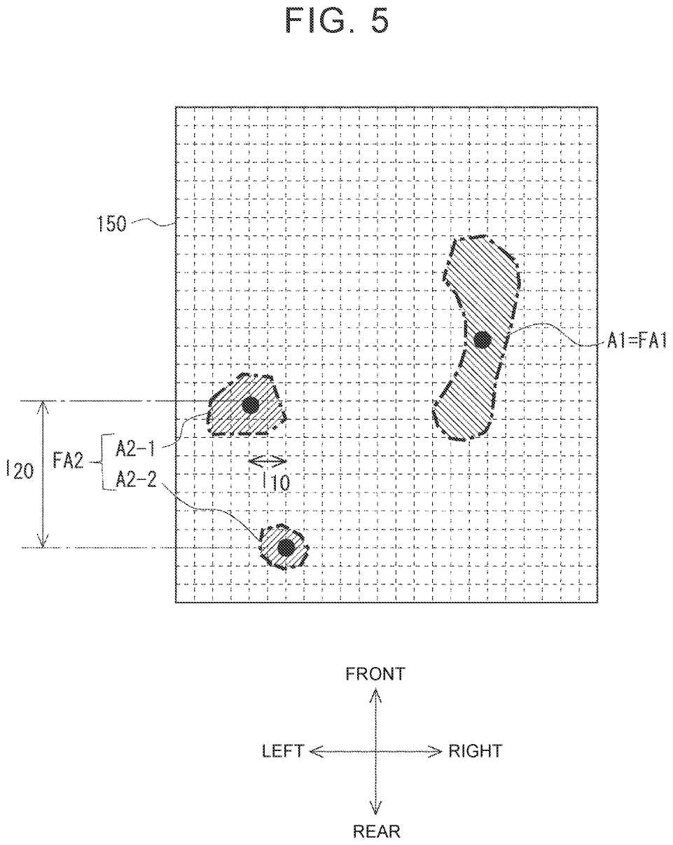

FIG. 5 is a diagram illustrating the first region according to the first embodiment. The first extraction unit 102 extracts the pressure detection points at which the load values equal to or larger than the first threshold value are detected, based on the load values detected at the pressure detection points and included in the measurement information. The first extraction unit 102 extracts a region A having a load value equal to or larger than the first threshold value from the arrangement region of the load distribution sensor 150 based on the positions of the extracted pressure detection points. Here, when only a single continuous region A1 is extracted as the region having a load value equal to or larger than the first threshold value, the first extraction unit 102 specifies the region A1 as a first region FA.

In contrast, when the first extraction unit 102 extracts a plurality of continuous regions as the regions having load values equal to or larger than the first threshold value (for example, regions A1, A2-1, A2-2), the first extraction unit 102 determines whether the region is generated by receiving a load from the same sole based on the position of each region A. Specifically, the first extraction unit 102 obtains the positions of the centers of gravity of the regions A, and clusters the regions A into clusters of two or less based on the distances between the centers of gravity of the regions A. Then, the first extraction unit 102 specifies each cluster as a first region FA.

For example, when the distance between the centers of gravity of two regions A in the right-left direction is less than 1₁, the first extraction unit 102 determines that the two regions A are generated by receiving a load from the same sole and classifies the two regions A into one cluster. Further, the first extraction unit 102 may determine that the two regions A are generated by receiving a load from the same sole, when the distance between the centers of gravity of two regions in the right-left direction is less than 1₁ and the distance in the walking front-rear direction of is less than 1₂. At this time, 1₂ may be longer than 1₁. In this example, a distance 1₁₀ between the centers of gravity of the regions A2-1 and A2-2 in the right-left direction is less than 1₁, and a distance 1₂₀ in the walking front-rear direction is less than 1₂. Therefore, the first extraction unit 102 classifies the region A2-1 and the region A2-2 into one cluster. With respect to the region A1, neither the region A2-1 nor the region A2-2 satisfies the determination condition for the distance between the centers of gravity. Therefore, the first extraction unit 102 classifies the region A1 into a cluster different from the cluster of the region A2-1 and the region A2-2. The first extraction unit 102 specifies the region A1 as the first region FA1 and the region A2-1 and the region A2-2 as the first region FA2.

When a plurality of regions A having load values equal to or larger than the first threshold value is classified into three or more clusters by clustering, the first threshold value may be too low and thus noise may be detected. Thus, the first extraction unit 102 may set the first threshold value higher by a predetermined value and re-extract the region A having a load value equal to or larger than the first threshold value.

The first extraction unit 102 provisionally specifies the specified first region FA as the sole region SL. Next, the second extraction unit 103 determines a re-extraction target region TA for each of the first regions FA specified by the first extraction unit 102.

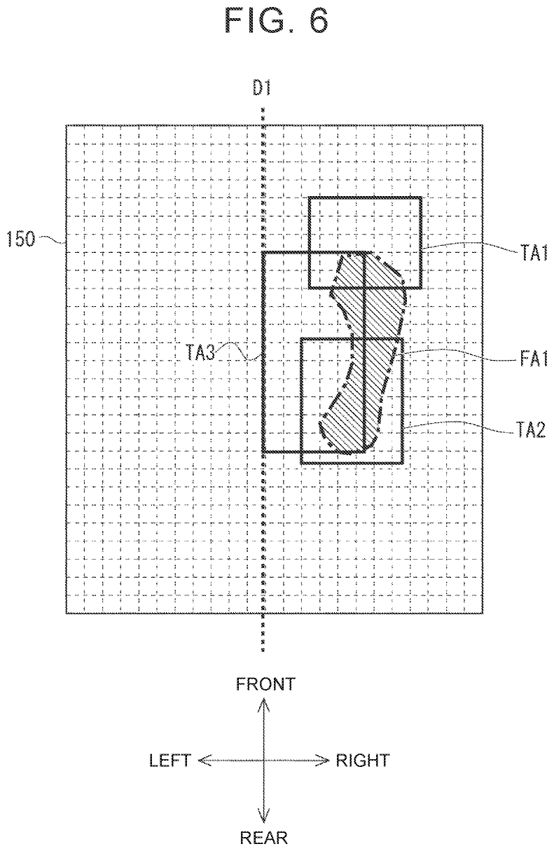

FIG. 6 is a diagram illustrating an example of the re-extraction target region TA. In FIG. 6, the first region FA1 described with reference to FIG. 5 is shown. Here, the second extraction unit 103 may determine the re-extraction target region TA based on the first region FA and the type of the re-extraction target.

For example, when the third input information indicates that the re-extraction target is the “toe”, the second extraction unit 103 determines the re-extraction target region TA1 as the re-extraction target region TA. The re-extraction target region TA1 is a region in which the front end portion in the walking direction protrudes forward in the walking direction of the trainee 900 from the front end portion of the first region FA1 by a predetermined distance. Alternatively, the re-extraction target region TA1 may be a region in which the position of the center of gravity is separated forward in the walking direction of the trainee 900 by a predetermined distance from the position of the center of gravity of the first region FA1. The position of the center of gravity of the re-extraction target region TA1 may coincide with the position of the center of gravity of the first region FA1 in the right-left direction. The distance between the position of the center of gravity of the re-extraction target region TA1 and the position of the center of gravity of the first region FA1 in the right-left direction may be less than a predetermined value. By defining the re-extraction target region TA1 in this way, the load measurement device 100 can easily detect the low load region corresponding to the toe.

The length of the re-extraction target region TA1 in the right-left direction may be determined based on the length of the first region FA1 in the right-left direction. The length of the re-extraction target region TA1 in the right-left direction may be larger than the length of the first region FA1 in the right-left direction. As an example, the length of the re-extraction target region TA1 in the right-left direction is n₁ times (n₁>1) the length of the first region FA1 in the right-left direction. The value of n₁ may be determined in advance.

The length of the re-extraction target region TA1 in the walking front-rear direction may be determined based on the length of the first region FA1 in the walking front-rear direction. The length of the re-extraction target region TA1 in the walking front-rear direction may be smaller than the length of the first region FA1 in the walking front-rear direction. As an example, the length of the re-extraction target region TA1 in the walking front-rear direction is m₁ times (m₁<1) the length of the first region FA1 in the walking front-rear direction. The value of m₁ may be determined in advance.

For example, when the third input information indicates that the re-extraction target is the “heel”, the second extraction unit 103 determines the re-extraction target region TA2 as the re-extraction target region TA. The re-extraction target region TA2 is a region in which the rear end portion in the walking direction protrudes rearward in the walking direction of the trainee 900 from the rear end portion of the first region FA1 by a predetermined distance. Alternatively, the re-extraction target region TA2 may be a region in which the position of the center of gravity is separated rearward in the walking direction of the trainee 900 by a predetermined distance from the position of the center of gravity of the first region FA1. The position of the center of gravity of the re-extraction target region TA2 may coincide with the position of the center of gravity of the first region FA1 in the right-left direction. The distance between the position of the center of gravity of the re-extraction target region TA2 and the position of the center of gravity of the first region FA1 in the right-left direction may be less than a predetermined value. By defining the re-extraction target region TA2 in this way, the load measurement device 100 can easily detect the low load region corresponding to the heel.

The length of the re-extraction target region TA2 in the right-left direction may be determined based on the length of the first region FA1 in the right-left direction. The length of the re-extraction target region TA2 in the right-left direction may be larger than the length of the first region FA1 in the right-left direction. As an example, the length of the re-extraction target region TA2 in the right-left direction is n₂ times (n₂>1) the length of the first region FA1 in the right-left direction. The value of n₂ may be determined in advance.

The length of the re-extraction target region TA2 in the walking front-rear direction may be determined based on the length of the first region FA1 in the walking front-rear direction. The length of the re-extraction target region TA2 in the walking front-rear direction may be smaller than the length of the first region FA1 in the walking front-rear direction. As an example, the length of the re-extraction target region TA2 in the walking front-rear direction is m₂ times (m₂<1) the length of the first region FA1 in the walking front-rear direction. The value of m₂ may be determined in advance.

For example, when the third input information indicates that the re-extraction target is the “arch of the foot”, the second extraction unit 103 determines a re-extraction target region TA3 as the re-extraction target region TA. Here, the position of the re-extraction target region TA3 with respect to the first region FA1 differs depending on whether the first region FA1 corresponds to the sole of the right leg or the sole of the left leg. Therefore, the second extraction unit 103 first determines whether the first region FA1 corresponds to the sole of the right leg or the sole of the left leg. For example, the second extraction unit 103 determines the leg including the sole that the first region FA1 corresponds to, based on the position of the first region FA1 with respect to the central axis D1 of the load distribution sensor 150 in the right-left direction. As an example, the second extraction unit 103 calculates the position of the center of gravity of the first region FA1. When the position of the center of gravity is located to the right of the central axis D1, the second extraction unit 103 may determine that the first region FA1 corresponds to the sole of the right leg.

Further, the second extraction unit 103 may determine whether the detected first region FA1 corresponds to the sole of the right leg or the sole of the left leg, based on the image obtained by capturing an image of the gait of the trainee 900 with the camera 140 serving as the front camera unit and the side camera unit. For example, when the captured image includes the trainee 900 with his/her sole of the right leg being in contact with the ground, the second extraction unit 103 determines that the detected first region FA1 corresponds to the sole of the right leg. Further, the second extraction unit 103 may determine whether the first region FA1 corresponds to the sole of the right leg or the sole of the left leg by estimating the positions of the soles corresponding to the legs of the trainee 900 from the captured image and specifying the sole that is estimated to be located closest to the position of the first region FA1.

When the soles of the two legs are in contact with the ground and two first regions FA are specified, the second extraction unit 103 may determine whether each first region FA corresponds to the sole of the right leg or the sole of the left leg based on the relative positions of the two first regions FA. For example, the second extraction unit 103 may determine, of the two first regions FA, a first region FA located on the right side as the first region FA corresponding to the sole of the right leg. Also in this case, the second extraction unit 103 may estimate the right and left of the first region FA based on the captured image.

When the second extraction unit 103 determines that the first region FA1 corresponds to the sole of the right leg, the second extraction unit 103 determines the re-extraction target region TA such that the left end portion of the re-extraction target region TA protrudes to the left by a predetermined distance from the left end portion of the first region FA. When the second extraction unit 103 determines that the first region FA1 corresponds to the sole of the left leg, the second extraction unit 103 determines the re-extraction target region TA such that the right end portion of the re-extraction target region TA protrudes to the right by a predetermined distance from the right end portion of the first region FA1. In FIG. 6, the first region FA1 corresponds to the sole of the right leg. Therefore, the second extraction unit 103 determines the re-extraction target region TA3 as the re-extraction target region TA. By defining the re-extraction target region TA3 in this way, the load measurement device 100 can easily detect the low load region corresponding to the arch of the foot.

In FIG. 6, the re-extraction target region TA is shown as a rectangle, but the shape of the re-extraction target region TA is not limited to a rectangle, and may be a circle, an ellipse, a half moon, a trapezoid, or any other shape.

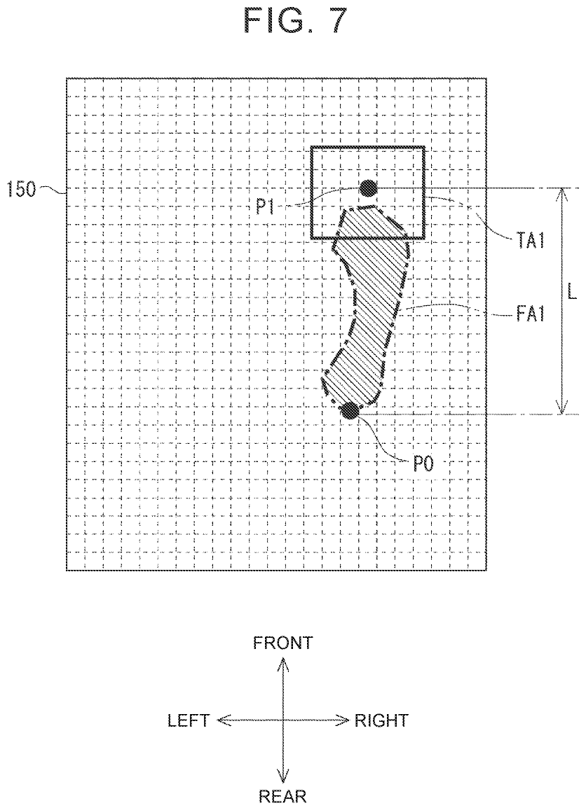

The second extraction unit 103 may determine the re-extraction target region TA based on the first region FA and the foot length of the trainee 900. In this case, the third input information may include the foot length information of the trainee 900. FIG. 7 is a diagram illustrating an example of the re-extraction target region TA according to the first embodiment. Also in FIG. 7, the first region FA1 described with reference to FIG. 5 is shown. The second extraction unit 103 specifies a point P0 located at the rear end portion of the first region FA1 in the walking direction as a position in the first region FA1 that corresponds to the heel. When the foot length of the trainee 900 is a length L, the second extraction unit 103 estimates a point P1 separated forward in the walking direction by the length L from the point P0 as the position of the toe.

Here, when the third input information indicates that the re-extraction target is the “toe”, the second extraction unit 103 specifies, as the re-extraction target region TA, the re-extraction target region TA1 having the point P1 as the center of gravity. The width, length, area, and shape of the re-extraction target region TA1 are the same as those described with reference to FIG. 6.

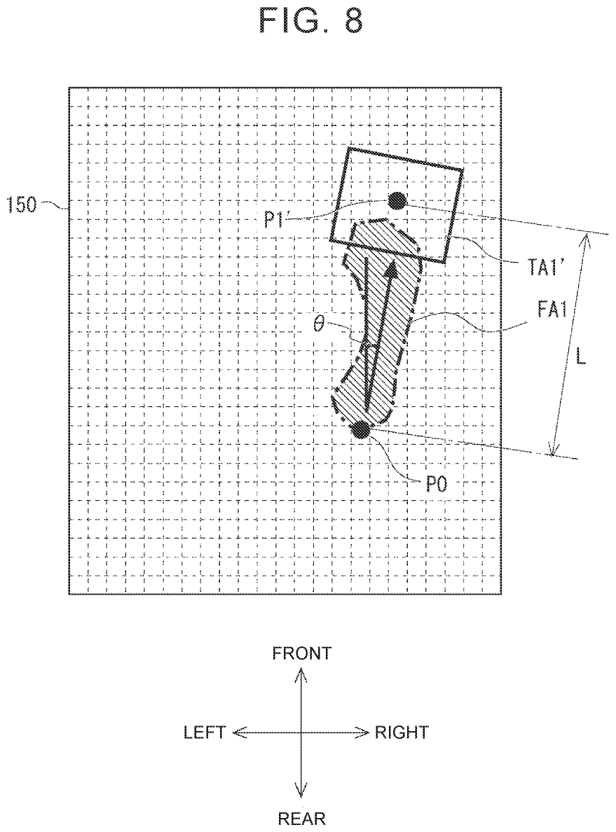

The second extraction unit 103 may estimate the orientation of the sole and determine the re-extraction target region TA based on the first region FA and the orientation of the sole. FIG. 8 is a diagram illustrating an example of the re-extraction target region TA according to the first embodiment. Also in FIG. 8, the first region FA1 described with reference to FIG. 5 is shown. The second extraction unit 103 estimates the orientation of the sole based on the morphological information of the lower leg. As an example, the morphological information of the lower leg may be bow legs (genu varum) or knock knees (genu valgum). In this case, the third input information may include the morphological information of the lower leg as the attribute information of the trainee 900. The second extraction unit 103 may estimate the central axis of the first region FA1 from the morphological information of the lower leg included in the third input information, and estimate the orientation of the sole based on the central axis. Further, the second extraction unit 103 may estimate the orientation of the sole based on the first region FA1. For example, the second extraction unit 103 may calculate the central axis of the first region FA1 by performing principal component analysis (PCA) on the position information of the first region FA1 and estimate the orientation of the sole based on the central axis. In FIG. 8, an angle θ between the central axis of the first region FA1 and the walking front-rear direction (traveling direction of the belt 132) is shown as the orientation of the sole.

The second extraction unit 103 determines the re-extraction target region TA based on the first region FA and the orientation of the sole. For example, the second extraction unit 103 determines, as the re-extraction target region TA, the re-extraction target region TA1′ obtained by tilting the re-extraction target region TA1 shown in FIG. 6 by the angle θ with respect to the walking front-rear direction.

The second extraction unit 103 may determine the re-extraction target region TA based on the foot length of the trainee 900 in addition to the first region FA and the orientation of the sole. For example, the second extraction unit 103 estimates that a point P1′ is the position of the heel. The point P1′ is separated forward in the direction of the central axis of the first region FA1 by the length L from the point P0 in the first region FA1 that corresponds to the heel. When the third input information indicates that the re-extraction target is the “toe”, the second extraction unit 103 specifies, as the re-extraction target region TA, the re-extraction target region TA1′ having the point P1′ as the center of gravity. The width, length, area, and shape of the re-extraction target region TA1′ are the same as those in the description of the re-extraction target region TA1.

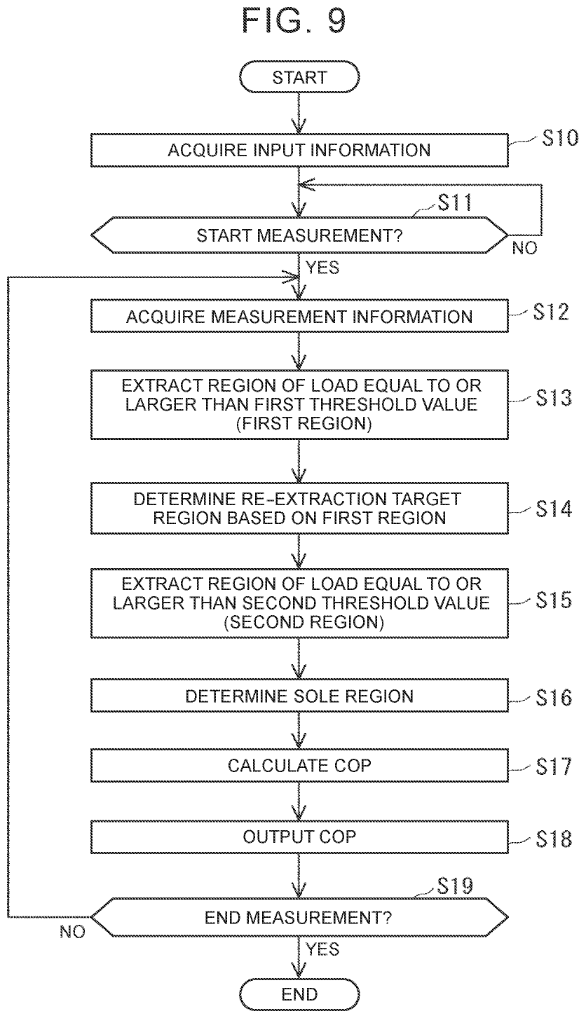

FIG. 9 is a flowchart showing a procedure of a load measurement method according to the first embodiment. First, the acquisition unit 101 acquires the input information received from the trainee 900 or the operator 910 at the input portion 142 (step S10). Next, the load measurement device 100 determines whether to start measurement (step S11). Examples of starting measurement include a case of starting training with the walking training system 1, a case of starting a load measurement process of the load measurement device 100 with the operation of the operator 910, and the like. The load measurement device 100 repeats the process shown in step S11 until it is determined to start the measurement. When it is determined to start the measurement (YES in step S11), the process proceeds to step S12.

In step S12, the acquisition unit 101 acquires the measurement information output by the load distribution sensor 150. Then, the first extraction unit 102 sets the first threshold value based on the first input information, and extracts (specifies) the first region FA that is a region having a load value equal to or larger than the first threshold value, from the measurement information (step S13). The first extraction unit 102 provisionally detects the first region FA as the sole region SL. Then, the second extraction unit 103 determines the re-extraction target region TA based on the first region FA and the third input information (step S14). The second extraction unit 103 sets the second threshold value based on the second input information. Then, the second extraction unit 103 extracts (specifies) a second region SA that is a region having a load value equal to or larger than the second threshold value, from the first region FA (step S15). The second extraction unit 103 determines the sole region SL by adding the second region SA to the provisionally detected sole region SL (step S16). When the second region SA is not extracted, the process shown in step S16 is omitted.

Then, the calculation unit 104 calculates the COP of the sole region SL based on the position information and the measurement information of the sole region SL (step S17). Then, the output unit 105 outputs the position information of the COP of the sole region SL to the system control unit 200 (step S18). Then, the load measurement device 100 determines whether to end the measurement (step S19). Examples of ending the measurement include a case of ending the training with the walking training system 1, a case of ending the load measurement process of the load measurement device 100 with the operation of the operator 910, and the like. The load measurement device 100 repeats the processes shown in steps S12 to S18 until it is determined to end the measurement.

As described above, according to the first embodiment, the load measurement device 100 scans the entire arrangement region of the load distribution sensor 150 with a predetermined threshold value, and then scans only a specific region with a lowered threshold value, thereby accurately detecting a low load region such as a toe while reducing the influence of noise. Thus, the measurement accuracy of the load distribution-related information of the sole region SL is improved. In particular, when the load measurement device 100 measures the COP of the sole region SL as the load distribution-related information of the sole region SL, errors of the COP position can be reduced. Thereby, the walking training system 1 can appropriately control the leg robot and carry out suitable walking training.

Further, the first threshold value and the second threshold value are set based on the attribute information of the trainee 900. Therefore, the load measurement device 100 can acquire a suitable load measurement result for each trainee 900. Thus, the measurement accuracy is improved.

### Second Embodiment

Next, a second embodiment of the present disclosure will be described. The load measurement device 100 according to the second embodiment has the same configuration as the load measurement device 100 according to the first embodiment. Therefore, the description thereof will be omitted. However, the load measurement device 100 according to the second embodiment is characterized in determining the re-extraction target region with a portion that the sole first comes into contact with as a reference.

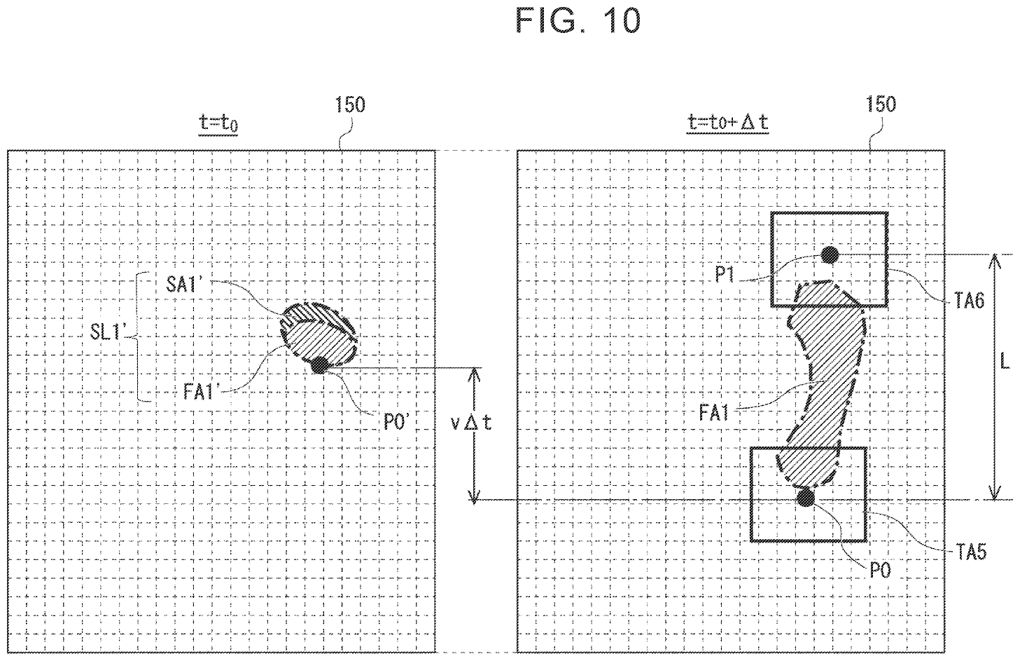

FIG. 10 is a diagram illustrating an example of the re-extraction target region TA according to the second embodiment. The left figure of FIG. 10 shows the sole region SL1′ included in the arrangement region of the load distribution sensor 150 at time t=t₀ that is the standing initial phase. The sole region SL1′ includes a first region FA1′ and a second region SA1′.

For example, when the area of the sole region SL of one leg tends to increase and becomes equal to or larger than a predetermined threshold value, the second extraction unit 103 may determine that the motion state of the leg is the standing initial phase. Alternatively, when the total load value of the sole region SL of one leg tends to increase and becomes equal to or larger than a predetermined threshold value, the second extraction unit 103 may determine that the motion state of the leg is the standing initial phase. When the second extraction unit 103 detects the standing initial phase, the second extraction unit 103 specifies a rear end portion or a rear end point of the sole region SL in the walking direction. In general, the trainee 900 often contacts the ground from the heel, so the rear end portion or the rear end point in the walking direction represents the position of the heel at the start of stepping. In the left figure of FIG. 10, the second extraction unit 103 specifies the point P0′ as the rear end point in the walking direction.

The right figure of FIG. 10 shows the first region FA1 and the re-extraction target region TA5 included in the arrangement region of the load distribution sensor 150 at time t=t₀+Δt after a predetermined time (Δt) has elapsed from the standing initial phase. Here, the second extraction unit 103 estimates the position of the heel at time t=t₀+Δt. Here, a case is considered in which the belt 132 of the treadmill 131 moves rearward in the walking direction at a predetermined speed v with respect to the arrangement region of the load distribution sensor 150. In this case, it can be estimated that the position of the heel of the trainee 900 at time t=t₀+Δt is located at the point P0 rearward, by vΔt, of the point P0′ that is the position of the heel at time t=t₀.

Then, the second extraction unit 103 determines the re-extraction target region TA based on the position of the heel at time t=t₀+Δt. For example, when the re-extraction target is the “heel”, the second extraction unit 103 may determine, as the re-extraction target region TA, the re-extraction target region TA5 having the position of the heel at time t=t₀+Δt (point P0) as the center of gravity.

Further, for example, when the re-extraction target is the “toe”, the second extraction unit 103 may determine the re-extraction target region TA6 as the re-extraction target region TA, based on the position of the heel at time t=t₀+Δt (point P0) and the foot length information of the trainee 900. The specific method for specifying the re-extraction target region TA6 may be the same as the method described with reference to FIG. 7 or FIG. 8.

The widths, lengths, areas, and shapes of the re-extraction target regions TA5 and TA6 are the same as those described above.

As described above, according to the second embodiment, the second extraction unit 103 determines the re-extraction target region TA based on the position information of the rear end portion or the rear end point of the sole region SL in the walking direction (heel) at the standing initial phase and the traveling speed of the treadmill 131 and the elapsed time from the standing initial phase. When the re-extraction target is the “toe”, the second extraction unit 103 determines the re-extraction target region TA based on the foot length information of the trainee 900 in addition to the above. That is, the second extraction unit 103 determines the re-extraction target region TA, with the portion that the heel first comes into contact with as a reference and using the attribute information of the trainee 900 as appropriate in accordance with the re-extraction target. This improves the estimation accuracy of the load measurement device 100 for the sole region SL.

The above description is based on the assumption that the trainee 900 contacts the ground from the heel. However, when the trainee 900 tends to contact the ground from the toe, the second extraction unit 103 may determine the re-extraction target region TA based on the position information of the front end portion or the front end point of the sole region SL in the walking direction (toe) at the standing initial phase and the traveling speed of the treadmill 131 and the elapsed time from the standing initial phase. In this case, when the re-extraction target is the “heel”, the second extraction unit 103 may determine the re-extraction target region TA based on the foot length information of the trainee 900 in addition to the above.

### Third Embodiment

Next, a third embodiment of the present disclosure will be described. The load measurement device 100 according to the third embodiment has the same configuration as the load measurement device 100 according to the first and second embodiments. Therefore, the description thereof will be omitted. However, the load measurement device 100 according to the third embodiment is characterized in changing the second threshold value during the walking training of the trainee 900.

Here, when the trainee 900 is a severe rehab patient, the gait of the trainee 900 is not stable. Therefore, if the same second threshold value is used throughout the measurement, there is a case where the low load region cannot be detected depending on the number of walk cycles. Thus, it is preferable to change the second threshold value in accordance with the stability of the gait. Specifically, the second extraction unit 103 may determine whether the gait is stable based on a history of at least one of the area, the shape, and the orientation of the sole region SL during the walking training of the trainee 900. For example, the second extraction unit 103 determines that the gait is not stable when the difference in the area, shape, or orientation of the sole region SL among the walk cycles is significant. As an example, the second extraction unit 103 compares the areas of the sole region SL after a predetermined time has elapsed from the standing initial stage, among the walking cycles. When the difference in area is equal to or greater than the predetermined threshold value, the second extraction unit 103 determines that the gait is not stable.

When the second extraction unit 103 determines that the gait is not stable, the second threshold value may be changed. For example, in this case, the second extraction unit 103 may reset the second threshold value to be smaller by a predetermined value. When the second extraction unit 103 determines that the gait is not stable and thereafter determines that the gait is stable, the second extraction unit 103 may estimate that the gait has recovered and change the second threshold value. For example, in this case, the second extraction unit 103 may reset the second threshold value to be larger by a predetermined value.

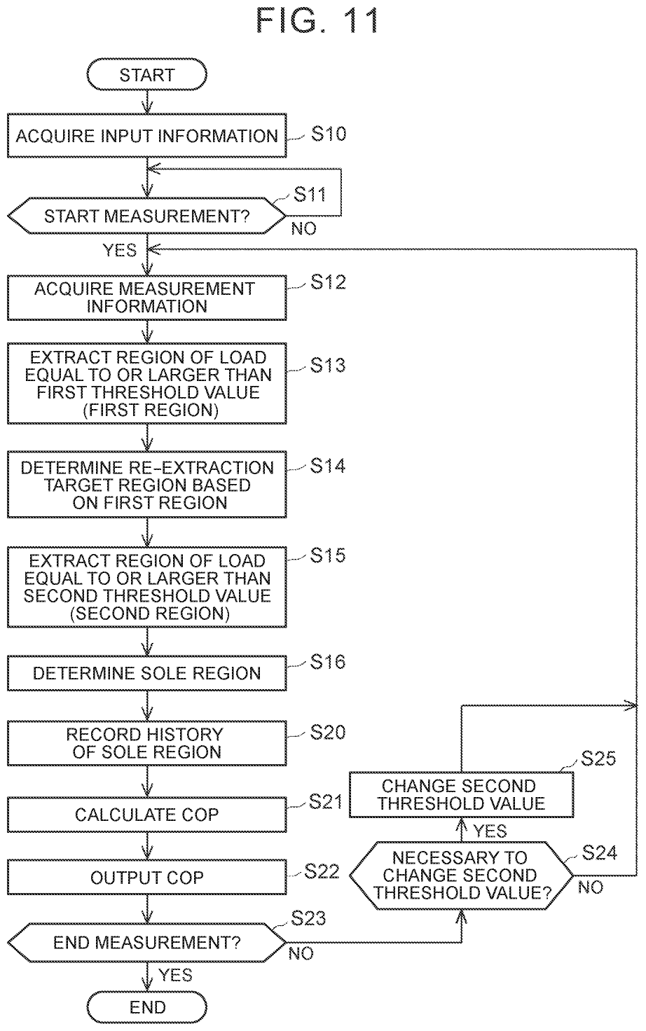

FIG. 11 is a flowchart showing a procedure of a load measurement method according to the third embodiment. The steps shown in FIG. 11 include steps S20 to S25 in addition to the steps shown in FIG. 9. The second extraction unit 103 records the history of the sole region SL in the storage unit 106 in response to the determination of the sole region SL in step S16 (step S20). Here, the history of the sole region SL may be the history of the area, shape, or orientation of the sole region SL. For example, the history of the sole region SL may include a walking cycle number, an elapsed time from the standing initial phase, and information on the area, shape, or orientation of the sole region SL. The walking cycle number is a number indicating the number of the walking cycle at the time of detection from the start of measurement.

Then, the load measurement device 100 executes steps S21 to S23 that are the same processes as steps S17 to S19. When the load measurement device 100 does not determine to end the measurement (NO in step S23), the second extraction unit 103 determines whether it is necessary to change the second threshold value (step S24). The second extraction unit 103 may determine whether it is necessary to change the second threshold value by determining whether the gait is stable by the method described above. When the second extraction unit 103 determines that it is necessary to change the second threshold value (YES in step S24), the second threshold value is changed (step S25), and the process returns to step S12. When the second extraction unit 103 determines that it is not necessary to change the second threshold value (NO in step S24), the process returns to step S12.

As described above, according to the third embodiment, the load measurement can be suitably performed even for the trainee 900 whose gait is not stable, and the measurement accuracy is improved.

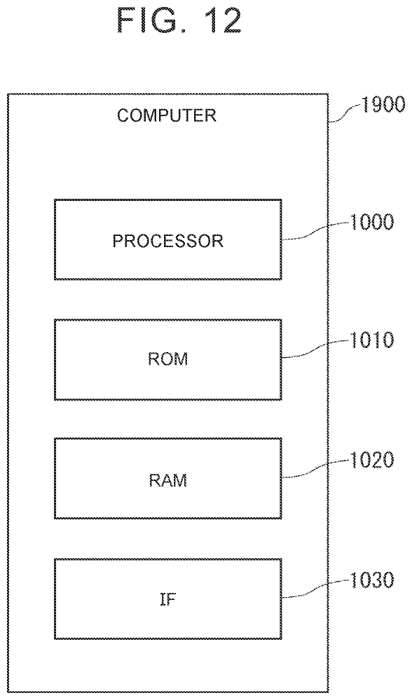

FIG. 12 is a schematic configuration diagram of a computer that is used as the load measurement device 100 and the system control unit 200 according to the first to third embodiments. A computer 1900 includes a processor 1000, a read only memory (ROM) 1010, a random access memory (RAM) 1020, and an interface unit 1030 (IF; interface) as main hardware configurations. The processor 1000, the ROM 1010, the RAM 1020 and the interface unit 1030 are connected to each other via a data bus and the like.

The processor 1000 has a function as an arithmetic unit that performs a control process, an arithmetic process, and the like. The processor 1000 may be a central processing unit (CPU), a graphics processing unit (GPU), a field-programmable gate array (FPGA), a digital signal processor (DSP), an application specific integrated circuit (ASIC), or a combination thereof. The ROM 1010 has a function for storing a control program, an arithmetic program, and the like executed by the processor 1000. The RAM 1020 has a function for temporarily storing processing data and the like. The interface unit 1030 inputs and outputs signals to and from the outside by wire or wirelessly. The interface unit 1030 receives the operation of inputting data by the user and displays information to the user. For example, the interface unit 1030 communicates with the load distribution sensor 150, the input portion 142, and the system control unit 200.

In the above example, the program includes instructions (or software codes) for causing the computer to perform one or more of the functions described in the embodiments when loaded into the computer. The program may be stored on various non-transitory computer-readable media or tangible storage media as an example of the ROM 1010. Examples of the computer-readable media or tangible storage media include, but not limited to, a RAM, a ROM, a flash memory, a solid-stated rive (SSD) or other memory technologies, a compact disc (CD)-ROM, a digital versatile disc (DVD), a Blu-ray (registered trademark) disc, or other optical disc storages, a magnetic cassette, a magnetic tape, a magnetic disc storage, or other magnetic storage devices. The program may be transmitted on a transitory computer-readable medium or a communication medium. Examples of the transitory computer-readable medium or the communication medium include, but not limited to, electrical, optical, acoustic, or other forms of propagating signals.

In the above embodiments, the computer 1900 is composed of a computer system including a personal computer, a word processor, and the like. However, the present disclosure is not limited thereto, and the computer 1900 can be composed of a local area network (LAN) server, a host computer of computer (personal computer) communication, a computer system connected on the Internet, and the like. The computer 1900 can be composed of the network as a whole with the functions being distributed to various devices on the network. Thus, the components of the load measurement device 100 may be distributed in different devices.

The present disclosure is not limited to the above embodiments, and can be appropriately modified without departing from the scope thereof. For example, the second extraction unit 103 may determine the re-extraction target in accordance with the stage of the walking cycle (standing initial phase, standing middle phase, and standing end phase). For example, the second extraction unit 103 may set the re-extraction target to the “toe” in the standing initial phase, to the “toe” and the “arch of the foot” in the standing middle phase, and to the “heel” in the standing end phase.

Further, in the third embodiment described above, the second extraction unit 103 changes the second threshold value based on the history of at least one of the area, the shape, and the orientation of the sole region SL during the walking training of the trainee 900. However, the first extraction unit 102 may also change the first threshold value based on the history of at least one of the area, the shape, and the orientation of the sole region SL during the walking training of the trainee 900, similarly to the change of the second threshold value by the second extraction unit 103.

In the above-described first to third embodiments, the load measurement device 100 is applied to the walking training system 1 including the treadmill 131. However, the present disclosure is not limited to this, and the load measurement device 100 may be applied to any other device including the load distribution sensor 150 that measures the load received from the sole of the trainee 900.

The trainee 900 may also perform training while wearing the walking assist device 120 on both legs. Alternatively, the trainee 900 does not have to wear the walking assist device 120 on any of the legs.

What is claimed is:

1. A load measurement system comprising:
an acquisition unit that acquires measurement information output from a load distribution sensor that detects a load distribution received from a sole of a subject;
a first extraction unit that extracts a region having a load value equal to or larger than a first threshold value as a first region based on the measurement information, and detects the first region as a sole region;
a second extraction unit that extracts a second region having a load value equal to or larger than a second threshold value that is smaller than the first threshold value from a re-extraction target region defined with the first region as a reference, and adds the second region to the sole region; and
an output unit that outputs information related to a load distribution of the sole region.

2. The load measurement system according to claim 1, further comprising a calculation unit that calculates a center of pressure of the sole region, wherein the output unit outputs information on the center of pressure.

3. The load measurement system according to claim 1, wherein the second extraction unit determines, as the re-extraction target region, a region located within a predetermined distance from a position of the first region.

4. The load measurement system according to claim 1, wherein the second extraction unit determines the re-extraction target region such that a front end portion of the re-extraction target region in a walking direction protrudes forward in the walking direction of the subject by a predetermined distance from a front end portion of the first region in the walking direction.

5. The load measurement system according to claim 1, wherein the second extraction unit determines the re-extraction target region such that a left end portion of the re-extraction target region protrudes to left by a predetermined distance from a left end portion of the first region when the first region is a sole region of a right leg, and determines the re-extraction target region such that a right end portion of the re-extraction target region protrudes to right by a predetermined distance from a right end portion of the first region when the first region is a sole region of a left leg.

6. The load measurement system according to claim 1, wherein the second extraction unit determines the re-extraction target region based on position information of a rear end portion of the sole region in a walking direction at a standing initial phase and foot length information of the subject.

7. The load measurement system according to claim 1, wherein the second extraction unit sets the second threshold value based on attribute information of the subject.

8. The load measurement system according to claim 1, wherein the second extraction unit changes the second threshold value based on a history of an area or a shape of the sole region during walking of the subject.

9. A walking training system comprising:
the load distribution sensor;
the load measurement system according to claim 1; and
a control device that controls extension of a leg robot attached to at least one leg of the subject based on the information related to the load distribution of the sole region of the subject.

10. A load measurement method comprising:
a step of acquiring measurement information output from a load distribution sensor that detects a load distribution received from a sole of a subject;
a step of extracting a region having a load value equal to or larger than a first threshold value as a first region based on the measurement information, and detecting the first region as a sole region;
a step of extracting a second region having a load value equal to or larger than a second threshold value that is smaller than the first threshold value from a re-extraction target region defined with the first region as a reference, and adding the second region to the sole region; and
a step of outputting information related to a load distribution of the sole region.

11. A program for causing a computer to execute a load measurement method, the load measurement method comprising:
a step of acquiring measurement information output from a load distribution sensor that detects a load distribution received from a sole of a subject;
a step of extracting a region having a load value equal to or larger than a first threshold value as a first region based on the measurement information, and detecting the first region as a sole region;
a step of extracting a second region having a load value equal to or larger than a second threshold value that is smaller than the first threshold value from a re-extraction target region defined with the first region as a reference, and adding the second region to the sole region; and
a step of outputting information related to a load distribution of the sole region.
